(19) United States
(12) Patent Application Publication
(10) Pub. No.: US 2007/0123573 A1
(43) Pub. Date: May 31, 2007

(54) Process for the preparation of biologically active tetrahydrobenzthiazole derivative

(75) Inventors: Dhiren N. Mistry, Vadodara (IN); Kamlesh S. Soni, Vadodara (IN); Sanjay L. Vasoya, Vadodara (IN); Vinod Kumar Kansal, Vadodara (IN)

Correspondence Address: HAMRE, SCHUMANN, MUELLER & LARSON, P.C., P.O. BOX 2902, MINNEAPOLIS, MN, 55402-0902, US

(73) Assignee: ALEMBIC LIMITED, Gujarat (IN)

(21) Appl. No.: 10/588,564

(22) PCT Filed: Apr. 25, 2005
(86) PCT No.: PCT/IN05/00127

§ 371 Date: Aug. 4, 2006

(30) Foreign Application Priority Data
Jul. 1, 2004 (IN) 706/MUM/2004

(51) Int. Cl. A61K 31/428; C07D 277/82

(52) U.S. Cl. 514/367; 548/161

(57) ABSTRACT

Improved process for the preparation of the intermediate compound of formula II for formation of biological active tetrahydrobenzothiazole compound of formula (I) as well as the biological active tetrahydrobenzothiazole compound of formula (I) and/or its pharmaceutically acceptable salts or solvates. 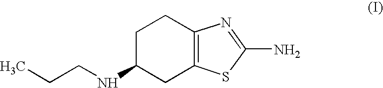 The process comprises reacting 4-amino cyclohexanol of formula (III) or its acid addition salts with phthalic anhydride in presence of acid catalyst and their salts, in polar aprotic solvent or its mixture with organic solvent, capable of removing water azeotropically to give 4-(phthalimido)-cyclohexanol of formula (IV); oxidizing 4-(phthalimido)-cyclohexanol of formula (IV) to give 4-(phthalimido)-cyclohexanone of formula (V); brominating 4-(phthalimido)-cyclohexanone of formula (V) with brominating agent in organic solvent in presence of Lewis acid catalyst to prepare 2-bromo-4-(phthalimido)-cyclohexanone of formula (VI); treating 2-bromo-4-(phthalimido)-cyclohexanone of formula (VI) with thiourea in organic solvent in presence of base to give 2-amino-6-phthalimido-4,5,6,7-tetrahydro benzothiazol of formula (VII); reacting compound of formula (VII) with hydrazine hydrate and base in polar solvent to give racemic 2,6-diamino-4,5,6,7-tetrahydro-1,3-benzothiazole of formula (VIII); resolving racemic 2,6-diamino-4,5,6,7-tetrahydro-1,3-benzothiazole of formula (VIII) to prepare (6S)-2,6-diamino-4,5,6,7-tetrahydro-1,3-benzothiazole of formula (II). To form the compound of Formula I and if desired its salts/solvates the above process is carried out with further steps of coupling (6S)-2,6-dimino-4,5,6,7-tetrahydro-1,3-benzothiazole of formula (II) with propionaldehyde in presence of mineral acid in polar organic solvent and reducing agent to prepare (S)-(−)-2-Amino-6-(n-propylamino)-4,5,6,7-tetrahydrobenzothiazole of formula (I);and if desired converting (S)-(−)-2-Amino-6-(propylamino)-4,5,6,7-tetrahydrobenzothiazole to its pharmaceutically acceptable salts or solvates.

### FIELD OF THE INVENTION

The present invention relates to an improved process for the preparation of (S)-(−)-2-Amino-6-(n-propylamino)-4,5,6,7-tetrahydrobenzothiazole of formula (I) and its pharmaceutically acceptable salts or solvates and (S)-2,6-diamino-4,5,6,7-tetrahydro benzothiazole an intermediate compound of formula II for formation of Pramipexole of Formula (1). The compound of formula I is commonly known as Pramipexole which is used in the chemotherapy of Parkinson's disease and schizophrenia. More particularly, the present invention is pertaining to an improved process for the preparation of Pramipexole dihydrochloride

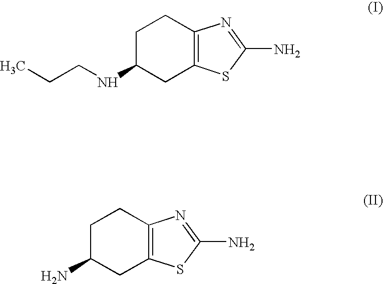

### BACKGROUND AND PRIOR ART

A general process for the preparation of compounds of formula (I) and (II) has been described in U.S. Pat. No. 4,886,812, EP 186087 and EP 207696. The process comprises the protection of amino function of 4-aminocyclohexanol (III) to give the compound of formula (IVa) wherein, R₁ is acyl or alkoxycarbonyl and R₂ is hydrogen or R₁ and R₂ together form an amino protective group such as pthalimido group which on oxidation with an oxidising agent, followed by halogenation (preferably bromination) of protected ketone of formula (Va) to give alpha halogenatedketone (VIa) which on reaction with thiourea, followed by deprotection yielded the racemic 2,6-diaminotetrahydrobenzothiole (VIIIa). Reductive alkylation of (VIIIa) with n-propanal furnished the racemic pramipexole. Although, the (S) isomer of pramipexole is mentioned therein, it is not clear at what stage the chiral resolution i.e. stage (VIII) or at final stage has been carried out. The general process steps are indicated in Scheme-1 below.

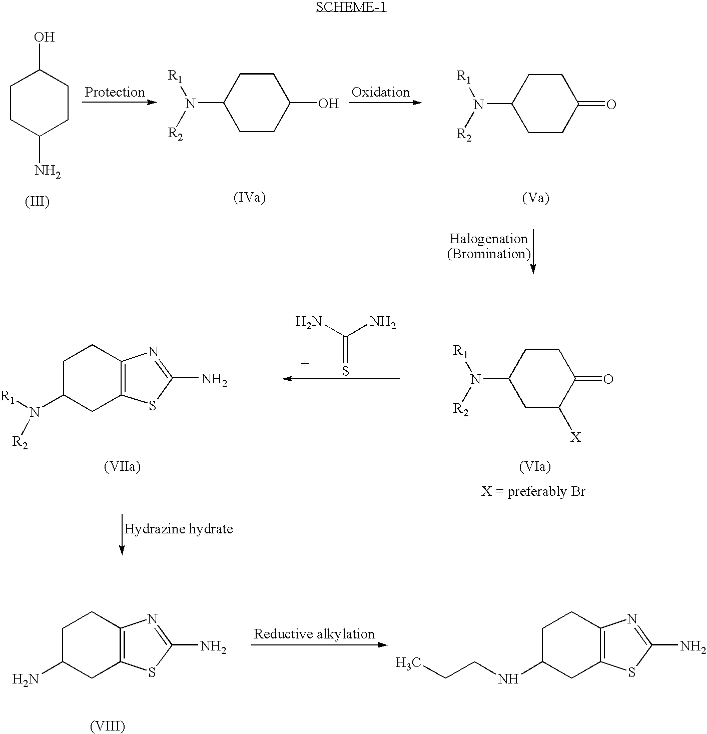

Another process for preparing optically pure pramipexole dihydrochloride was disclosed in J Med. Chem.1987, 30,494-498 , where in, racemic 2,6-diamino-4,5,6,7-tetrahydro 5 benzothiazole was resolved, using L(+)tartaric acid to give optically pure (S)-2,6-diamino-4,5,6,7-tetrahydro benzothiazole which was converted to optically pure pramipexole by reacting (S)-2,6-diamino-4,5,6,7-tetrahydro benzothiazole with propionic anhydride in THF and followed by reduction with borane THF complex. The reaction steps are shown in Scheme-2 as under:

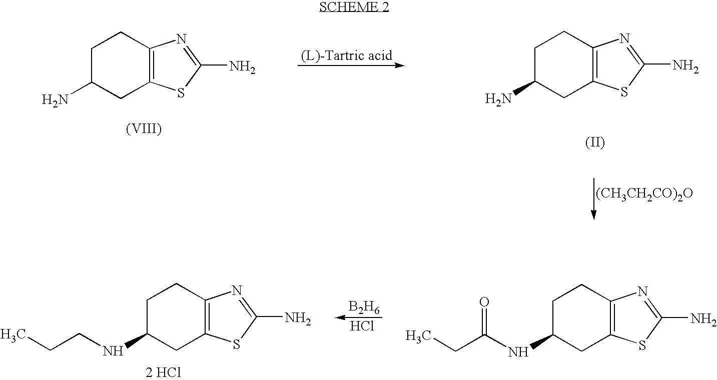

The processes described above, suffer with the following drawbacks:
(i) Although, phthalamido protected 4-aminocyclohexanole gives better yield compared to monoprotected 4-aminocyclohexanol during oxidation and halogenation, the protection of 4-aminocyclohexanol with phthalic anhydride requires longer duration, approximately 36 hrs, hence will increase utility, manpower & overall cost of production. Furthermore, the efforts to repeat the reaction in the reported conditions were futile.
(ii) Bromination is carried out with hydrobromic acid in acetic acid, which is corrosive in nature. Work up of the reaction is very tedious. Moreover, diethyl ether has been used to remove the impurities. Diethyl ether is highly flammable and has low flash point, hence paused fire hazards at commercial scale.
(iii) Moreover, 2-amino-6-phthalimido-4,5,6,7-tetrahydro benzothiazole requires laborious column chromatography to isolate and purify the 2-amino-6-phthalimido-4,5,6,7-tetrahydro benzothiazole. Use of column chromatography is not feasible at commercial scale and gives low yield i.e. 50%.
(iv) Yet another disadvantage of the process lies in preparation and isolation of 2,6-diamino-4,5,6,7-tetrahydro benzothiazole dihydrochloride as it also requires column chromatography and give very poor yield i.e. 26%.

Overall, the process disclosed in U.S. Pat. No. 4,886,812, EP 186087 and EP 207696 for the preparation of pramipexole, are lengthy, low yielding, requires laborious column chromatography for several steps and use of corrosive and highly flammable materials. Therefore, there is a need to develop a process for preparing pramipexole and its pharmaceutically acceptable salts, solvates, which should be free from the above mentioned defects and should be simple, cost effective, high yielding and does not involve laborious column chromatography. Also, process should be devoid of highly flammable and corrosive material for commercial production.

### OBJECTS OF THE INVENTION

Thus one object of the invention is to provide an improved process for the preparation of (S)-2,6-diamino-4,5,6,7-tetrahydro benzothiazole of formula (II), which is a key intermediate for the synthesis of Pramipexole.

Another object of this invention is to provide an improved process for the preparation of pramipexole of formula (I) and its pharmaceutically acceptable salts, solvates if desired free from the above-mentioned defects.

Another object of this invention is to provide commercially viable process for the preparation of pramipexole and its pharmaceutically acceptable salts, solvates.

Yet another object of the process is to reduce the time of condensation of phthalic anhydride with 4-aminocyclohexanole.

Yet another object of the process is to simplify the work up of halogenation without using flammable solvent.

Yet another object of the invention is to provide a process for the preparation of Pramipexole, devoid of column chromatography at every stage of the process.

Further object of the invention is to overcome the problems associated with prior art process and to prepare Pramipexole by cost effective way.

In summary, the object of the present invention is to provide a simple, efficient, cost effective, devoid of corrosive, highly inflammable material, high yielding process for the preparation of Pramipexole of formula (I) and its pharmaceutically acceptable salts, solvates.

### SUMMARY OF THE INVENTION

Thus according to one aspect of present invention, there is provided an improved process for the preparation of (S)-2,6-diamino-4,5,6,7-tetrahydro benzothiazole of formula (II), an intermediate compound for formation of Pramipexole of Formula (I) and its pharmaceutically acceptable salts, solvates 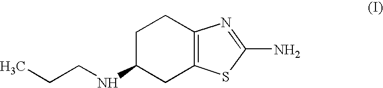
comprising the steps of
(a) reacting 4-amino cyclohexanol of formula (III) or its acid addition salts with phthalic anhydride in presence of acid catalyst and their salts, in polar aprotic solvent or its mixture with organic solvent, capable of removing water azeotropically to give 4-(phthalimido)-cyclohexanol of formula (IV) 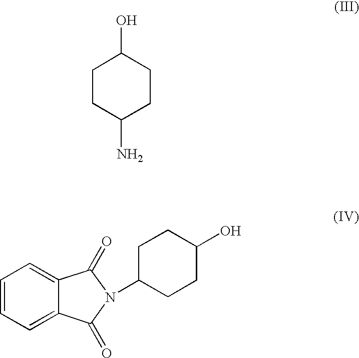
(b) oxidizing 4-(phthalimido)-cyclohexanol of formula (IV) to give 4-(phthalimido)-cyclohexanone of formula (V) 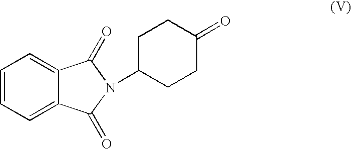
(c) brominating 4-(phthalimido)-cyclohexanone of formula (V) with brominating agent in organic solvent in presence of Lewis acid catalyst to prepare 2-bromo-4-(phthalimido)-cyclohexanone of formula (VI) 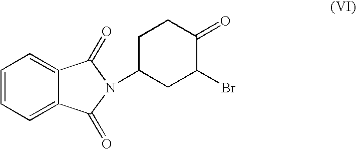
(d) treating 2-bromo-4-(phthalimido)-cyclohexanone of formula (VI) with thiourea in organic solvent in presence of base to give 2-amino-6-phthalimido-4,5,6,7-tetrahydro benzothiazol of formula (VII) 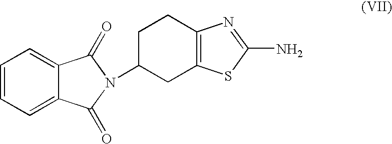
(e) reacting compound of formula (VII) with hydrazine hydrate and base in polar solvent to give racemic 2,6-diamino-4,5,6,7-tetrahydro-1,3-benzothiazole of 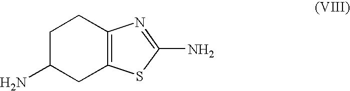 formula (VIII)
(f) resolving racemic 2,6-diamino-4,5,6,7-tetrahydro-1,3-benzothiazole of formula (VIII) to prepare (6S)-2,6-diamino-4,5,6,7-tetrahydro-1,3-benzothiazole of formula (II)

According to another aspect of present invention, there is provided an improved process for the preparation of Pramipexole of Formula (I) and its pharmaceutically acceptable salts/solvates 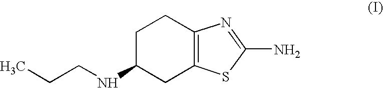
comprising the steps of
(a) reacting 4-amino cyclohexanol of formula (III) or its acid addition salts with phthalic anhydride in presence of acid catalyst and their salts, in polar aprotic solvent or its mixture with organic solvent, capable of removing water azeotropically to give 4-(phthalimido)-cyclohexanol of formula (IV) 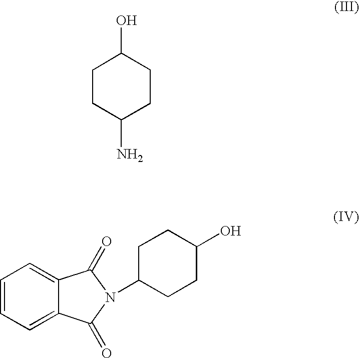
(b) oxidizing 4-(phthalimido)-cyclohexanol of formula (IV) to give 4-(phthalimido)-cyclohexanone of formula (V) 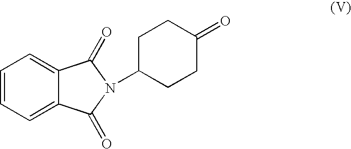
(c) brominating 4-(phthalimido)-cyclohexanone of formula (V) with brominating agent in organic solvent in presence of Lewis acid catalyst to prepare 2-bromo-4-(phthalimido)-cyclohexanone of formula (VI) 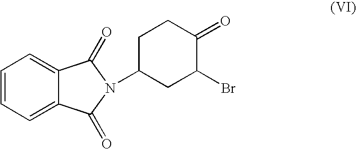
(d) treating 2-bromo-4-(phthalimido)-cyclohexanone of formula (VI) with thiourea in organic solvent in presence of base to give 2-amino-6-phthalimido-4,5,6,7-tetrahydro benzothiazol of formula (VII) 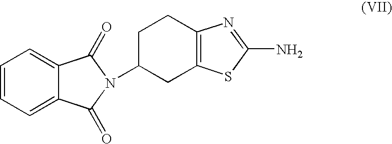
(e) reacting compound of formula (VII) with hydrazine hydrate and base in polar solvent to give racemic 2,6-diamino-4,5,6,7-tetrahydro-1,3-benzothiazole of formula (VIII) 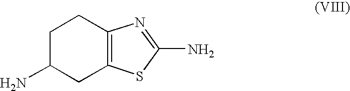
(f) resolving racemic 2,6-diamino-4,5,6,7-tetrahydro-1,3-benzothiazole of formula (VIII) to prepare (6S)-2,6-diamino-4,5,6,7-tetrahydro-1,3-benzothiazole of formula (II) 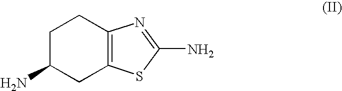
(g) coupling (6S)-2,6-dimino-4,5,6,7-tetrahydro-1,3-benzothiazole of formula (II) with propionaldehyde in presence of mineral acid in polar organic solvent and reducing agent to prepare (S)-(−)-2-Amino-6-(n-propylamino)-4,5,6,7-tetrahydrobenzothiazole of formula (I); and if desired
(h) converting (S)-(−)-2-Amino-6-(propylamino)-4,5,6,7-tetrahydrobenzothiazole to its pharmaceutically acceptable salts or solvates.

### DETAILED DESCRIPTION OF THE INVENTION

According to an improved process for the preparation of Pramipexole of Formula (I) and its pharmaceutically acceptable salts, solvates when desired is shown in SCHEME-3 as follows:

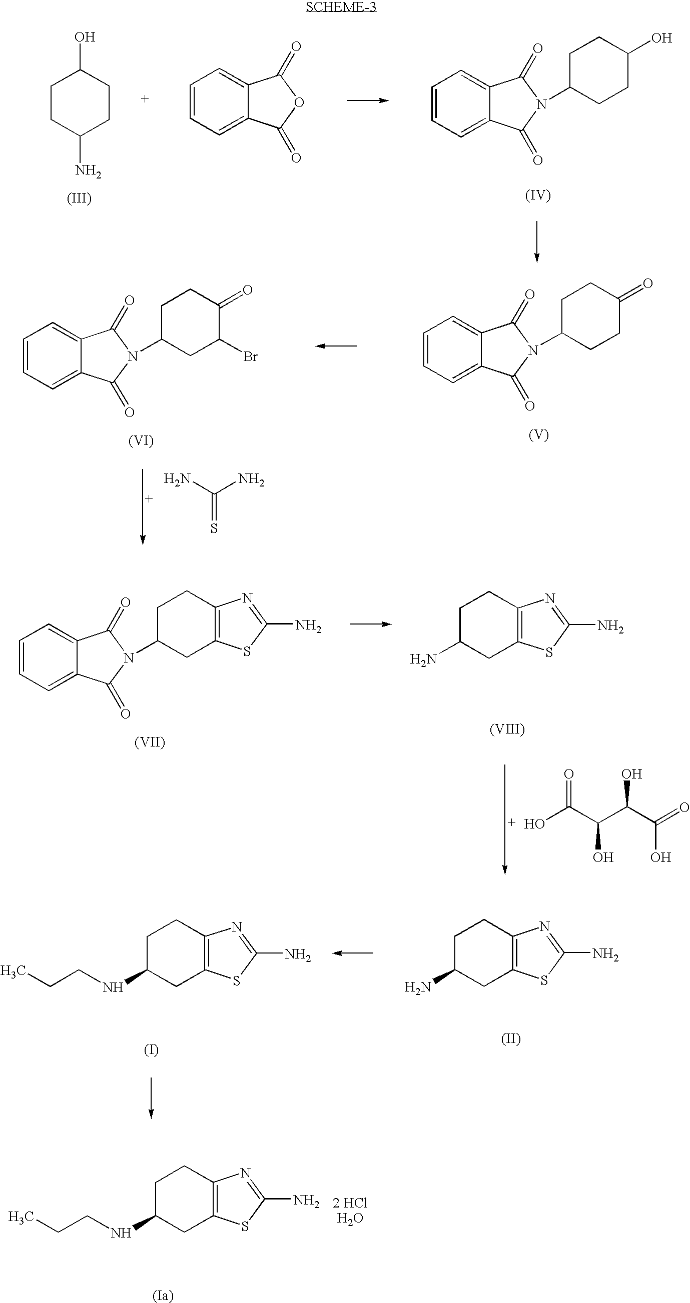

According to the present invention, 4-amino cyclohexanol of formula (III) is reacted with pthalic anhydride in presence of acid catalyst or their salts with organic bases, in polar aprotic solvent or its mixture with organic solvents, capable of removing water azeotropically.

Acid catalysts used in step (a) are sulphonic acid and their salts with organic bases and salt of inorganic acids with organic bases. It is selected form the group comprising of p-toluene sulphonic acid (PTSA), methane sulphonic acid, acid addition salts of pyridine, picoline, lutidine such as pyridine hydrochloride, pyridine hydrobromide, pyridine methane sulfonate, pyridine p-toluene sulphonate, picoline hydrochloride, picoline hydrobromide, picoline methane sulphonate, picoline p-toluene sulphonate, lutidine hydro chloride, lutidine hydrobromide, lutidine methane sulphonate, lutidine p-toluene sulphonate. The preferred acid catalyst is p-toluene sulphonic acid, pyridine p-toluene sulphonate

Polar aprotic solvent used in above step (a) is selected from group comprising of amide functional group such as dimethylformamide (DMF), dimethylacetamide (DMAC), N-methylpyrrolidinone (NMP), N-methylacetamide, N-methylformamide, N,N-dimethylpropionamide, sulphoxide functional group such as dimethylsulfoxide, sulfolane, and ethers such as tetrahydrofuran (THF) and dioxane,

The preferred solvent is dimethyl formamide. Also, step (a) can be carried out in mixture of polar aprotic solvent with organic solvent, capable of removing water azeotropically such as toluene, cyclohexane and the like. The preferred organic solvent is selected from toluene, cyclohexane.

Reaction step (a) is carried out at 90° C. to 140° C. for 10 to 20 hrs and preferably for 12 to 18 hrs.

4-(phthalimido)-cyclohexanol of formula (IV) is further oxidized by conventional manner to give 4-(phthalimido)-cyclohexanone of formula (V). (4-phthalimido)-cyclohexanol is oxidized with potassium dichromate and H₂SO₄ to give 4-(phthalimido)-cyclohexanone.

4-(phthalimido)-cyclohexanone is further brominated with brominating agent in presence of Lewis acid as catalyst in organic solvent and converted to 2-amino-6-phthalimido-4,5,6,7-tetrahydro benzothiazole with thiourea.

Brominating agent used in step (c) is bromine and a Lewis acid catalyst is selected from the group comprising of aluminium chloride, zinc chloride, stannous chloride.

Bromination can be carried out in both halogenated and non halogenated organic solvents. Most preferred halogenated solvent is selected from methylene dichloride, most preferred non halogenated solvents are alkyl acetate such as ethyl acetate, methyl acetate, propyl acetate and alcohols such as methanol, ethanol, and propanol. Step (c) is carried out at −5 to 40° C. and more preferably at 0° C. to 10° C.

2-bromo-4-(phthalimido)-cyclohexanone of formula (VI) is with or without isolating and is further treated with thiourea in presence of base in organic solvent to give 2-amino-6-phthalimido-4,5,6,7-tetrahydro benzothiazole. Base used in step (d) is selected from alkaline earth metal carbonate, bicarbonates and acetate. Preferred base is selected from sodium carbonate, potassium carbonate, sodium bicarbonate, potassium bicarbonate, and sodium acetate and potassium acetate. The most preferred base used in step (d) is sodium bicarbonate or potassium bicarbonate.

Step (d) is carried out in organic solvent selected form alcohols, halogenated solvent or mixture there of. Alcohols is selected from methanol, ethanol, isopropanol, n-propanol, n-butanol or mixture there of. Halogenated solvent is selected form methylene dichloride, ethylene dichloride, chloroform.

2-amino-6-phthalimido-4,5,6,7-tetrahydrobenzothiazole of formula (VII) can also be prepared according to step (d) without isolating 2-bromo-4-(phthalimido)-cyclohexanone prepared in step (c). 2-bromo-4-(phthalimido)-cyclohexanone prepared by step (c) can be treated in situ with thoiurea in presence of base to give compound of formula (VII).

Reacting 2-amino-6-phthalimido-4,5,6,7-tetrahydro-benzothiazole of formula (VII) with hydrazine hydrate in presence of organic base in polar solvent to give racemic 2,6-diamino-4,5,6,7-tetrahydro benzothiazole (VIII). Moreover, 2,6-diamino-4,5,6,7-tetrahydro benzothiazole of formula (VIII) can also be isolated as its acid addition salts.

Organic base used in step (e) is selected from triethyl amine, pyridine, dimethyl aniline, lutidines, picolines and DBU. The preferred base used in step (e) is triethyl amine.

Polar solvent used in step (e) is selected form alcohols preferably methanol, ethanol, isopropanol, n-propanol, n-butanol, iso-butanol. The preferred solvent used in step (e) is ethanol or isopropanol.

Reaction step (e) is carried out at reflux temperature of above solvent

According to an important aspect of the invention, racemic 2,6-diamino-4,5,6,7-tetrahydro-benzothiazole of formula (VIII) prepared in step (e) is without isolating, further converted to its desired isomer (S)-2,6-diamino-4,5,67-tetrahydro-benzthiazole of formula (II)

Resolution of racemic 2,6-diamino-4,5,6,7-tetrahydro benzothiazole of formula (VIII) with L-tartaric acid lead to desired (S) isomer of 2,6-diamino-4,5,6,7-tetrahydro benzothiazole of formula II. Resolution of compound (VIII) comprises
(i) treating in situ or after isolating racemic 2,6-diamino-4,5,6,7-tetrahydro benzothiazole of formula (VIII), obtained in step (d) with (L)-tartric acid to give (S) tartrate salts of 2,6-diamino-4,5,6,7-tetrahydro benzothiazole
(ii) isolating pure (S) tartrate salts of 2,6-diamino-4,5,6,7-tetrahydro benzothiazole
(iii) converting pure (S) tartrate salts of 2,6-diamino-4,5,6,7-tetrahydro benzothiazole to (S)-2,6-diamino-4,5,6,7-tetrahydro benzothiazole of formula (II)

Reacting (S)-2,6-diamino-4,5,6,7-tetrahydro benzothiazole of formula (II) with propionaldehde in suitable organic solvent in presence of mineral acid and reducing agent leads to formation of pramipexole of formula (I).

Mineral acid used in step (g) is selected from hydrochloric acid, sulfuric acid. Preferred mineral acid is sulfuric acid. Reducing agent used in step (g) is metal borohydride.

Preferred metal borohydride is selected from sodium borohydride, sodium cyanoborohydride. Preferred reducing agent is sodium borohydride

Organic solvent used in step (g) is polar organic solvent preferably alcohols selected from methanol, ethanol, isopropanol and n-propanol.

Step (g) is carried out at 0° C. to 50° C. more preferably at 0° C. to 30° C.

Pramipexole of formula (I) is further converted to its pharmaceutically acceptable salt/solvates by reacting with the respective acid in solvent selected from ethyl acetate, isopropyl acetate, methanol, ethanol or mixtures there of The preferred salt is Pramipexole dihydrochloride, which is available in the market, is prepared by reacting pramipexole with hydrochloric acid or HCI gas in solvent to give Pramipexole dihydrochloride. Also, its solvate, i.e. Pramipexole dihydrochloride monohydrate is prepared by addition of water during salt formation.

The process of the present invention leads to a significantly increase in yield at all the steps and does not involved column chromatography. Furthermore, the bromination and cyclization reaction steps have been carried out without using corrosive material. The reagent used in presence of catalyst provides a significant increase in yield from 50% to 90% without using column chromatography.

Thus the present invention provides an efficient process for the preparation of pramipexole of formula (I) and its pharmaceutically acceptable salts, solvates, which offers significant commercial advantages when preparing on an industrial scale. The present invention is having several advantages over known process.

The process of the present invention produces pramipexole of formula (I) and more particularly pramipexole dihydrochloride monohydrate is simple, environment friendly and economical and leads to an enhanced yield.

The current process further provides significant efficiencies at the commercial manufacturing. The overall cost and labor of the manufacturing process are reduced, as simpler machinery can be used, simple method is involved and fewer undesirable waste products are generated, all of which provides distinct commercial advantages for the preparation of Pramipexole on a commercial scale.

The process of the present invention is described by the following examples, which are illustrative only and should not be construed so as to limit the scope of the invention in any manner.

### EXAMPLES

### Example-1

Preparation of 4-(phthalimido)-cyclohexanol

(A) 300 gms (2.608 mole) of Trans-4-aminocyclohexanol was dissolve in 1500 ml Dimethyl formamide and 1500 ml of Toluene. Add 386 gms(2.608 mole) of Phthalic anhydride and 3 gm(0.012 mole) pyridinium p-toluene sulphonate. The reaction mixture is refluxed and remove water continuously from water separator, maintain this condition for 15-17 hrs. Evaporate solvent under reduced pressure. Add chloroform (3000 ml). Wash organic part with 1000 ml of 5% NaHCO₃, then wash with 1000 ml of brine solution. After concentration of reaction mass, crystallize residue in Isopropyl alcohol.

YIELD : 503 gms (79%) PURITY: 99.66%

(B) 25 gms(0.2123 mole) Trans-4-aminocyclohexanol was dissolve in 100 ml cyclohexane and 100 ml DMF. Add 128.6 gm(0.8689 mole) phthalic anhydride and 0.25 gm(0.001 mole) pyridinium p-toluene sulphonate. Reflux mass at 90-95° C. for 19 hrs. Remove continuously water from water separator. Cool mass to 40° C., remove solvent under reduced pressure. Dissolve mass in 250 ml chloroform, washed chloroform layer with 5% NaHCO₃ solution and brine solution. Evaporate chloroform and residue was crystallizing in isopropyl alcohol.

YIELD: 38 gms(71%)

(C) 25 gms (0.2123 mole) Trans-4-aminocyclohexanol was dissolved in 125 ml of toluene and 125 ml of DMF. Add 32.17 gm(0.2123 mole) phthalic anhydride and 0.25 gm (0.0066 mole) of p-toluene sulphonic acid. Reflux mass at 130°-135° C. for 10 hrs. Remove continuously water from water separator. Cool mass to 40° C. remove solvent under reduced pressure. Dissolve mass in 250 ml chloroform, washed chloroform layer with 5% NaHCO3 solution and brine solution. Evaporate chloroform and residue was crystallizing in isopropyl alcohol.

YIELD: 41 gms(77%)

(D) 25 gms(0.2123 mole) Trans-4-aminocyclohexanol was dissolve in 125 ml of toluene and 125 ml of DMF. Add 32.17 gm(0.2123 mole) phthalic anhydride and 0.25 gm(0.0074 mole) of pyridine hydrobromide. Reflux mass at 130°-135° C. for 15-17 hrs. Remove continuously water from water separator. Cool mass to 40° C. remove solvent under reduced pressure. Dissolve mass in 250 ml chloroform, washed chloroform layer with 5% NaHCO₃ solution and brine solution. Evaporate chloroform and residue was crystallizing in isopropyl alcohol.

YIELD: 37 gms(69.4%)

### Example: 2

Preparation of 4-(phthalimido)-cyclohexanone

190 gms(0.7755 mole) 4-phthalimido cyclohexanol are dissolve in 1480 ml chloroform. Add solution of H₂SO₄ (435.87 gm, 4.4476 mole conc. H₂SO₄ was added in 900 ml water). Cool mass to 25° C., add lot wise 180.5 gm(0.6139 mole) potassium dichromate in one hour. Stir mass for three hours, add 900 ml water and separate organic phase. Organic phase was washed with water and 2% NaHCO3 solution, after drying and concentration of extracts product was isolated by adding methanol and water mixture.

YIELD: 175 g(92.4%) PURITY: 96.01%.

### Example: 3

Preparation of 3-bromo-4-(Phthalimido)-cyclohexanone

(A) 15 gm (0.0617 mole) 4-phthalimido cyclohexanone was dissolve in 150 ml methanol. Heat the mass to 40° C. Add Br₂ solution (9.8 gm Br₂ in 25 ml of methanol) and 0.25 gm of AlCl₃ under stirring. Stop stirring and allow initiating bromination and finding clear solution then add remaining quantity of Br₂ solution and stir for 10-15 mins. Add 10 ml water and stir for 10 mins more. Then filter the white solids obtain. Dry it at 50° C. for 2-3 hrs.

YIELD: 12.5 gm(62.8%)

(B) 15 gm (0.0617 mole) 4-phthalimido cyclohexanone was dissolve in 150 ml Ethyl acetate. Cool the mass to 0° C. Add Br₂ solution (9.8 gm Br₂ in 25 ml of methanol) and 0.25 gm of AlCl₃ under stirring. Stop stirring and allow initiating bromination and finding clear solution then add remaining quantity of Br₂ solution and stir for 10-15 mins. Wash the reaction mass with 75 ml 2% NaS₂O₃ solution then wash organic phase with 75 ml 8% NaHCO₃. Then in last wash it with brine solution. Collect organic masses and evaporate it under vacuum. Dry it at 50° C. for 2-3 hrs.

YIELD: 15 gms(75.2%)

### Example-4

Preparation of 2-amino-6-phthalimido-4,5,6,7-tetrahydro benzothiazole

100 gm(0.4115 mole) 4-phthalimido cyclohexanone was dissolve in 1000 ml dichloromethane. Cool the mass to 0° C. Add 25 ml Br₂ solution (65.8 gm Br₂ in 100 ml of dichloromethane) and 0.3 gm anhydrous AlCl₃ under stirring. Stop stirring and allow initiating bromination and finding clear solution then add remaining quantity of Br₂ solution and stirr for 10-15 min. Wash the reaction mass with 250 ml 2% NaS₂O₃ solution then wash organic phase with 250 ml 8% NaHCO₃. Collect organic phase and add 46 gm (0.6052 mole) thiourea, 34 gm (0.4047 mol) NaHCO₃ and 350 ml methanol. Reflux reaction mass for 2-3 hrs. Distill off dichloromethane and methanol. Add 690 ml DM water in residue. Filter the product and purified wet product by hot methanol.

YIELD: 110 gm(89%) PURITY: 96.45%

### Example-5

Preparation of Racemic 2,6-diamino-4,5,6,7-tetrahydro benzothiazole

100 gm(0.3344 mole) 2-amino-6-phthalimido-4,5,6,7-tetrahydro benzothiazole was suspended in 500 ml isopropyl alcohol. Add 20.05 gm(0.4010 mole) hydrazine hydrate and 7.26 gm (0.0718 mole) Triethylamine. Reflux for 2-3 hrs. Cool the mass to 10° C., Filter the slurry and wash with chilled isopropyl alcohol. Isolated mixture of compounds are recrystalize in absolute alcohol

YIELD: 50 gm(88.46%)

### Example-6

Preparation of (S)-Tartarate salt of 2,6-diamino-4,5,6,7-tetrahydro benzothiazole

100 gm (0.5917 mol) of 4,5,6,7-tetrahydro-1,3-benzothiazole-2,6-diamine was added in 1000 ml DM water. Heat it to 70° C. and add 88.75 gm (0.5917 mole) L(+)-Tartaric acid. Stirr for 1.5 hr, cool to 60° C. Filter hot. Stir the filtrate for 10-12 hrs, cool to 5° C. Stir for 30 mins. Filter and recrystallize by water.

PURITY: 99.5% (chiral purity)

### Example-7

Preparation of (S)-2,6-diamino-4,5,6,7-tetrahvdro benzothiazole

100 gm(0.3134 mole) (S)-Tartarate salt of 2,6-diamino-4,5,6,7-tetrahydro benzothiazole was added to 79.69 ml water. Cool the reaction mass to 0-5° C. with stirring. Add 71.99 ml conc. HCI slowly and drop wise. Then add 240 ml 85% KOH solution drop wise to reaction mass. Maintain temperature 0-5° C. during complete addition. Stir reaction mass for 1-2 hrs at 0-5° C. Filter the product.

YIELD: 56 gm(1.05%) PURITY: 99.6%

### Example-8

Preparation of (S)-(−)-2-Amino-6-(n-propylamino)-4,5,6,7-tetrahydrobenzothiazole or Pramipexole of formula (I)

50 gm (0.2958 mole) (6S)-4,5,6,7-tetrahydro-1,3-benzthiazole-2,6-diamine was dissolved in 1500 ml methanol. Bring down temp of solution to 0° C. Add 20.68 gm(0.3566 mole) propionaldehyde and 1.3 gm conc. sulfuric acid (0.044 mole). After stirring to 90 minutes add 16.78 gm (0.4435 mole) sodium borohydride. Allow increasing temperature of mass to 25° C. After one hrs add second lot of 17.22 gm (0.2970 mole) propionaldehyde and agitate for 10-15 mins. Then add 11.19 gm (0.2957 mole) sodium borohydride and stirr for 40 mins. Add 150-ml brine solution and stirr for 30 mins. Distill off solvent under reduced pressure at 40° C. Add 500 ml ethyl acetate and water, Separate organic phase, dry it and distilled off ethyl acetate under reduced pressure at 40° C. Residue is crystallizing in Acetonitrile.

YIELD: 34.75 gm (80.1%) PURITY: 99.5% ¹H NMR in DMSO: 1.14 ppm (d, 3H) C(3′); 4.12 ppm (m, 1H) C(2′); 3.0 ppm (m,1H) C(1′); 3.54 ppm (m,lH) C(6); 3.10 ppm (m, 2H) C(7); 2.34 ppm (m, 2H) C(3); 2.09 ppm (m, 2H) C(4) 13C NMR in DMSO: C(4) 23.2 ppm, C(5) 20.9 ppm, C(7) 24.69 ppm,. C(6) 52.65 ppm, C(1′) 51.51, C(2′) 62.30, C(3′) 21.02 ppm; thiazole ring C(2′) 168.7 ppm; C(4′) 132.8 ppm, C(5′) 110.83 ppm

### Example-9

Preparation of Pramipexole dihydrochloride monohydrate

100 gm (0.4739 mole) (S)-Pramipexole was dissolve in 800 ml ethanol. Heat it to 50-55° C. Add lOgm-charcoal powder and stirr for 15-20 min. Filter through hyflow and wash it with 200 ml ethanol. Add 8.53 gm (0.4739 mole) water cool the reaction mass to 0-5° C. Pass dry HCl gas to reaction mass till pH becomes 2. Stir for 7-8 hrs. Filter the product. Purified by refluxing with ethanol.

YIELD : 127 gm(88.7%) PURITY: 99.8%

While the present invention has been described in terms of its specific embodiments, certain modifications and equivalents will be apparent to those skilled in the art and are intended to be included within the scope of the present invention.

1. An improved process for the preparation of (S)-2,6-diamino-4,5,6,7-tetrahydro benzothiazole of formula 11 an intermediate compound for formation of Pramipexole of Formula (I) and its pharmaceutically acceptable salts, solvates 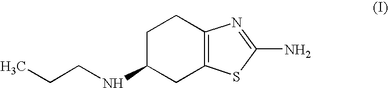
comprising the steps of
(a) reacting 4-amino cyclohexanol of formula (III) or its acid addition salts with phthalic anhydride in presence of acid catalyst and their salts, in polar aprotic solvent or its mixture with organic solvent, capable of removing water azeotropically to give 4-(phthalimido)-cyclohexanol of formula (IV) 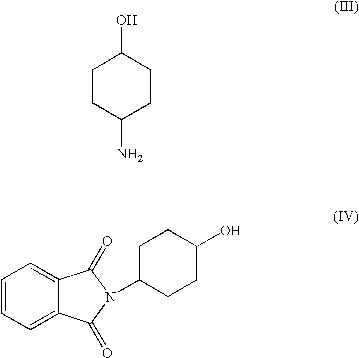
(b) oxidizing 4-(phthalimido)-cyclohexanol of formula (IV) to give 4-(phthalimido)-cyclohexanone of formula (V) 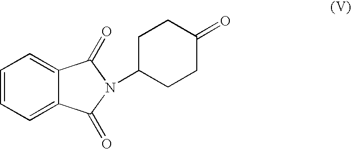
(c) brominating 4-(phthalimido)-cyclohexanone of formula (V) with brominating agent in organic solvent in presence of Lewis acid catalyst to prepare 2-bromo-4-(phthalimido)-cyclohexanone of formula (VI) 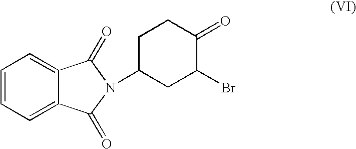
(d) treating 2-bromo-4-(phthalimido)-cyclohexanone of formula (VI) with thiourea in organic solvent in presence of base to give 2-amino-6-phthalimido-4,5,6,7-tetrahydro benzothiazol of formula (VII) 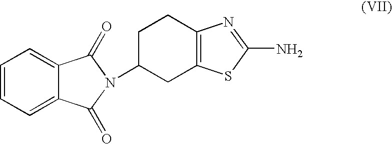
(e) reacting compound of formula (VII) with hydrazine hydrate and base in polar solvent to give racemic 2,6-diamino-4,5,6,7-tetrahydro-1,3-benzothiazole of formula (VIII) 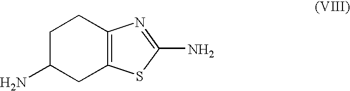
(f) resolving racemic 2,6-diamino-4,5,6,7-tetrahydro-1,3-benzothiazole of formula (VIII) to prepare (6S)-2,6-diamino-4,5,6,7-tetrahydro-1,3-benzothiazole of formula (II)

2. An improved process for the preparation of Pramipexole of Formula (I) and its pharmaceutically acceptable salts/solvates 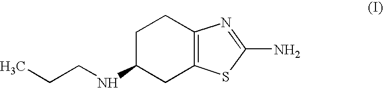
comprising the steps of
(a) reacting 4-amino cyclohexanol of formula (III) or its acid addition salts with phthalic anhydride in presence of acid catalyst and their salts, in polar aprotic solvent or its mixture with organic solvent, capable of removing water azeotropically to give 4-(phthalimido)-cyclohexanol of formula (IV) 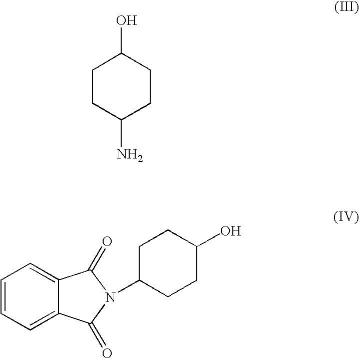
(b) oxidizing 4-(phthalimido)-cyclohexanol of formula (IV) to give 4-(phthalimido)-cyclohexanone of formula (V) 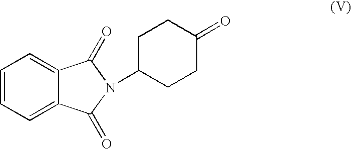
(c) brominating 4-(phthalimido)-cyclohexanone of formula (V) with brominating agent in organic solvent in presence of Lewis acid catalyst to prepare 2-bromo-4-(phthalimido)-cyclohexanone of formula (VI) 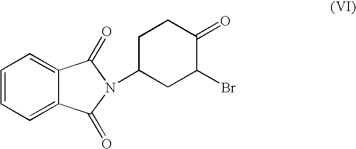
(d) treating 2-bromo-4-(phthalimido)-cyclohexanone of formula (VI) with thiourea in organic solvent in presence of base to give 2-amino-6-phthalimido-4,5,6,7-tetrahydro benzothiazol of formula (VII) 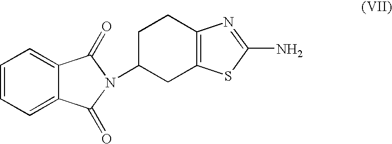
(e) reacting compound of formula (VII) with hydrazine hydrate and base in polar solvent to give racemic 2,6-diamino-4,5,6,7-tetrahydro-1,3-benzothiazole of formula (VIII) 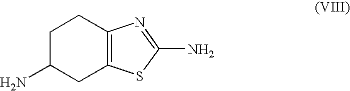
(f) resolving racemic 2,6-diamino-4,5,6,7-tetrahydro-1,3-benzothiazole of formula (VIII) to prepare (6S)-2,6-diamino-4,5,6,7-tetrahydro-1,3-benzothiazole of formula (II) 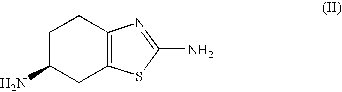
(g) coupling (6S)-2,6-dimino-4,5,6,7-tetrahydro-1,3-benzothiazole of formula (II) with propionaldehyde in presence of mineral acid in polar organic solvent and reducing agent to prepare (S)-(−)-2-Amino-6-(n-propylamino)-4,5,6,7-tetrahydrobenzothiazole of formula (I); and if desired
(h) converting (S)-(−)-2-Amino-6-(propylamino)-4,5,6,7-tetrahydrobenzothiazole to its pharmaceutically acceptable salts or solvates.

3. A process as claimed in claim 1, wherein acid catalyst in step (a) is sulphonic acid and its salts with organic bases or salt of inorganic acids with organic bases.

4. A process as claimed in claim 1, wherein said acid catalyst is selected form the group comprising of p-toluene sulfonic acid, methane sulfonic acid, pyridine hydrochloride, pyridine hydrobromide, pyridine methane sulfonate, pyridine p-toluene sulphonate, picoline hydrochloride, picoline hydrobromide, picoline methane sulfonate, picoline p-toluene sulphonate, lutidine hydro chloride, lutidine hydrobromide, lutidine methane sulfonate, lutidine p-toluene sulphonate.

5. A process as claimed in claim 4, wherein said acid catalyst is preferably pyridine p-toluene sulphonate, p-toluene sulfonic acid.

6. A process as claimed in claim 1, wherein said polar aprotic solvent in step (a) is selected from group comprising of amide functional group such as dimethylformamide (DMF), dimethylacetamide (DMAC), N-methylpyrrolidinone (NMP), N-methylacetamide, N-methylformamide, N,N-dimethylpropionamide, sulphoxide functional group such as dimethylsulfoxide, sulfolane, and ethers such as tetrahydrofuran (THF) and dioxane.

7. A process as claimed in claim 6, wherein preferred solvent is Dimethyl formamide.

8. A process as claimed in claim 1, wherein step (a) is carried out in mixture of polar aprotic solvent with organic solvent, capable of removing water azeotropically such as toluene, cyclohexane and the like

9. A process as claimed in 1, wherein said step (a) is carried out at 90° C. to 140° C.

10. A process as claimed in claim 1, wherein said step (a) is carried out for 10 to 20 hrs and more preferably for 12 to 18 hrs.

11. A process as claimed in claim 1, wherein brominating agent in said step (c) is bromine.

12. A process as claimed in claim 1, wherein Lewis acid used as catalyst in said step (c) is selected form aluminum chloride zinc chloride and stannous chloride.

13. A process as claimed in claim 12, wherein Lewis acid catalyst is preferably aluminum chloride

14. A process as claimed in claim 1, wherein organic solvent in said step (c) is selected from halogenated, nonhalogenated organic solvents.

15. A process as claimed in claim 14, wherein said halogenated solvent is methylene dichloride.

16. A process as claimed in claim 14, wherein said nonhalogenated solvents is selected from alkyl acetate such as ethyl acetate, methyl acetate, propyl acetate and alcohols such as methanol, ethanol, and propanol.

17. A process as claimed in claim 1, wherein base used in step (d) is selected from alkaline earth metal carbonate, bicarbonate, acetate.

18. A process as claimed in claim 17, wherein base is selected from sodium carbonate, potassium carbonate, sodium bicarbonate, potassium bicarbonate, sodium acetate, potassium acetate, preferably sodium bicarbonate, potassium bicarbonate.

19. A process as claimed in claim 1, wherein organic solvent used in step (d) is selected from alcohols, halogenated solvents or mixtures thereof.

20. A process as claimed in claim 19, wherein organic solvent used in step (d) is selected from methanol, ethanol, isopropranol, n-propanol, n-butanol, methylene dichloride, ethylenedichloride, chloroform, or mixtures thereof.

21. A process as claimed in claim 1, wherein said step (d) can be carried out without isolating 2-bromo-4-(phthalimido)-cyclohexanone of formula (VI) prepared in said step (c).

22. A process as claimed in 1, wherein said step (d) is carried out in situ with thiourea.

23. A process as claimed in claim 1, wherein organic base used in said step (e) is triethyl amine, pyridine, dimethy aniline, lutidines, picolines and DBU, preferably triethyl amine.

24. A process as claimed in claim 1, wherein said polar solvent in step (e) is selected from methanol, ethanol, isopropanol, n-propanol, n-butanol, iso-butanol or mixtures thereof.

25. A process as claimed in claim 24, wherein preferred solvent is ethanol or isopropanol.

26. A process as claimed in claim 1, wherein said step (f) comprises the steps of
(i) treating in situ or racemic 2,6-diamino-4,5,6,7-tetrahydro benzothiazole of formula (VIII), obtained in step (d) with (L) -tartric acid to give (S) tartrate salts of 2,6-diamino-4,5,6,7-tetrahydro benzothiazole.
(ii) isolating pure (S) tartrate salts of 2,6-diamino-4,5,6,7-tetrahydro benzothiazole
(iii) converting pure (S) tartrate salts of 2,6-diamino-4,5,6,7-tetrahydro benzothiazole to (S)-2,6-diamino-4,5,6,7-tetrahydro benzothiazole of formula (II).

27. A process as claimed in claim 2, where in mineral acid used in said step (g) is selected from HCI, H₂SO4 preferably H₂SO₄

28. A process as claimed in claim 2, wherein reducing agent used in said step (g) is metal borohydride preferably sodium borohydride, sodium cyanoborohydride.

29. A process as claimed in claim 2, wherein polar organic solvent used in step (g) is selected from alcohols preferably methanol, ethanol, isppropanol, n-propanol or mixtures thereof.

30. A process as claimed in claim 2, wherein the conversion of Pramipexole of Formula (I) to its pharmaceutically acceptable salts, solvates is carried out with respective acids in organic solvent selected from methanol, ethanol, ethyl acetate, isopropyl acetate.

31. A process for the preparation of (S)-2,6-diamino-4,5,6,7-tetrahydro benzothiazole an intermediate compound of formula II for formation of Pramipexole of Formula (I) such as herein described with particular reference to the examples.

32. A process for the preparation of pramipexole of formula (I) and its pharmaceutically acceptable salts solvates as herein described particularly with reference to the examples.

33. A process as claimed in claim 2, wherein acid catalyst in step (a) is sulphonic acid and its salts with organic bases or salt of inorganic acids with organic bases.

34. A process as claimed in claim 2, wherein said acid catalyst is selected form the group comprising of p-toluene sulfonic acid, methane sulfonic acid, pyridine hydrochloride, pyridine hydrobromide, pyridine methane sulfonate, pyridine p-toluene sulphonate, picoline hydrochloride, picoline hydrobromide, picoline methane sulfonate, picoline p-toluene sulphonate, lutidine hydro chloride, lutidine hydrobromide, lutidine methane sulfonate, lutidine p-toluene sulphonate.

35. A process as claimed in claim 2, wherein said polar aprotic solvent in step (a) is selected from group comprising of amide functional group such as dimethylformamide (DMF), dimethylacetamide (DMAC), N-methylpyrrolidinone (NMP), N-methylacetamide, N-methylformamide, N,N-dimethylpropionamide, sulphoxide functional group such as dimethylsulfoxide, sulfolane, and ethers such as tetrahydrofuran (THF) and dioxane.

36. A process as claimed in claim 2, wherein step (a) is carried out in mixture of polar aprotic solvent with organic solvent, capable of removing water azeotropically such as toluene, cyclohexane and the like

37. A process as claimed in 2, wherein said step (a) is carried out at 90° C. to 140° C.

38. A process as claimed in claim 2, wherein said step (a) is carried out for 10 to 20 hrs and more preferably for 12 to 18 hrs.

39. A process as claimed in claim 2, wherein brominating agent in said step (c) is bromine.

40. A process as claimed in claim 2, wherein Lewis acid used as catalyst in said step (c) is selected form aluminum chloride zinc chloride and stannous chloride.

41. A process as claimed in claim 2, wherein organic solvent in said step (c) is selected from halogenated, nonhalogenated organic solvents.

42. A process as claimed in claim 2, wherein base used in step (d) is selected from alkaline earth metal carbonate, bicarbonate, acetate.

43. A process as claimed in claim 2, wherein organic solvent used in step (d) is selected from alcohols, halogenated solvents or mixtures thereof.

44. A process as claimed in claim 2, wherein said step (d) can be carried out without isolating 2-bromo-4-(phthalimido)-cyclohexanone of formula (VI) prepared in said step (c).

45. A process as claimed in 2, wherein said step (d) is carried out in situ with thiourea.

46. A process as claimed in claim 2, wherein organic base used in said step (e) is triethyl amine, pyridine, dimethy aniline, lutidines, picolines and DBU, preferably triethyl amine.

47. A process as claimed in claim 2, wherein said step (f) comprises the steps of
(i) treating in situ or racemic 2,6-diamino-4,5,6,7-tetrahydro benzothiazole of formula (VIII), obtained in step (d) with (L) -tartric acid to give (S) tartrate salts of 2,6-diamino-4,5,6,7-tetrahydro benzothiazole.
(ii) isolating pure (S) tartrate salts of 2,6-diamino-4,5,6,7-tetrahydro benzothiazole
(iii) converting pure (S) tartrate salts of 2,6-diamino-4,5,6,7-tetrahydro benzothiazole to (S)-2,6-diamino-4,5,6,7-tetrahydro benzothiazole of formula (II).
